(19) United States
(12) Patent Application Publication
(10) Pub. No.: US 2008/0021003 A1
(43) Pub. Date: Jan. 24, 2008

(54) EXTENDED STEP-DOWN ESTROGEN REGIMEN

(76) Inventors: Vladimir Hanes, Tarrytown, NY (US); Jan-Peter Ingwersen, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/761,616

(22) Filed: Jun. 12, 2007

Related U.S. Application Data
(60) Provisional application No. 60/812,986, filed on Jun. 13, 2006.
(60) Provisional application No. 60/908,794, filed on Mar. 29, 2007.

(51) Int. Cl. A61K 31/56; A61P 5/24

(52) U.S. Cl. 514/170; 514/177

(57) ABSTRACT

The present invention relates to methods for treating diseases, conditions or symptoms associated with deficient endogenous levels of estrogen comprising administration of a higher first dose of an estrogen followed by administration of a lower second dose of an estrogen once therapy has been effectively established. The invention further relates to a combination treatment comprising administration of an estrogen and a progestin.

This application claims the benefit of the filing date of U.S. Provisional Application Ser. No. 60/812,986 filed Jun. 13, 2006 and U.S. Provisional Application Ser. No. 60/908,794 filed Mar. 29, 2007, which is incorporated by reference herein.

### FIELD OF INVENTION

This invention relates to methods for treating diseases, conditions or symptoms associated with deficient endogenous levels of estrogen comprising administration of a higher first dose of an estrogen followed by administration of a lower second dose of an estrogen once therapy has been effectively established. The invention further relates to a combination treatment comprising administration of an estrogen and a progestin.

### BACKGROUND

Estrogen deficiency in the perimenopausal and menopausal woman is manifested by both short-term symptoms and long-term system diseases. Menopause typically occurs in women during middle age and is usually associated with short-term symptoms including hot flushes, mood changes, urogenital changes, such as dryness and atrophy of the vagina, sexual dysfunction, and skin changes. Long-term, estrogen deficiency accelerates the risk of chronic diseases such as osteoporosis and cardiovascular disease.

Hot flushes are the most common and bothersome clinical symptom of menopause, affecting approximately 75% of postmenopausal women. The increase in occurrence of hot flushes is linked with the reduction of estrogen levels that go along with menopause. Menopausal symptoms cause discomfort and distress, ranging from tolerable to, at times, severe enough to affect one's quality of life. Currently, there are more than 40 million menopausal women in the US and almost half of them are over the age of 65. As life expectancy continues to increase, most women will spend one-third of their lifetime in menopause.

“Estrogen Replacement Therapy” has been used for several decades for the treatment of estrogen deficiency and has been established as an effective and safe treatment of moderate to severe vasomotor symptoms associated with menopause. However, one of the risks associated with the administration of estrogens is that women with intact uteri develop endometrial hyperplasia referring to over-stimulation of the lining of the uterus, which is a precursor to endometrial or uterine cancer. The development of endometrial hyperplasia is a significant side-effect of Estrogen Replacement Therapy.

It has been shown that progestins can reduce the development of endometrial hyperplasia induced by estrogen therapy. However, side effects often still occur with progestin co-administration. Thus, it is still desirable to have an estrogen replacement therapy in which potential side effects relating to the therapy are reduced.

At present, the lowest estrogen dose and regimen that will control vasomotor symptoms are recommended. However, administration of the lowest dose to begin estrogen replacement therapy often does not treat severe vasomotor symptoms.

Some investigations suggest that a high dose of estrogen (e.g. 1 mg estradiol orally/day) is necessary as starting dose to treat menopausal symptoms, however, a lower dose of estrogen (e.g. 0.5 mg estradiol orally/day) could be used after the initial therapy, and even a lower dose of estrogen can be administered then as a maintenance dose (e.g. 0.3 mg estradiol orally/day). Thus, a step-down estrogen regimen could be the most appropriate way to treat the menopausal symptoms over a long time period.

Results of studies such as the Women's Health Initiative (WHI) have intensified the need to investigate lower doses of estrogen for the treatment of menopausal symptoms due to safety concerns. In this respect, it is important to development regimens which would gradually reduce the estrogen dose to a maintenance dose which would then be used over a longer period without long term safety concerns which were shown by the WHI study. The need to adjust the estrogen dose according to individual responses during therapy is another reason for the proposed step-down estrogen regimen.

Drospirenone (DRSP), a progestin with anti-aldosterone activity has been developed for continuously combined hormone therapy in combination with the estrogen, 17β-estradiol (E2), in menopausal women (daily administration of DRSP/E2). The product is approved in the US, EU and other countries worldwide.

The following documents describe as step-down estrogen therapy optionally including administration of a progestin:

WO 03/084547 discloses methods for treating vasomotor symptoms through the administration of estrogenic compounds, including starting estrogen therapy at a high dose and then lowering the dose once therapy is effective. A progestational agent may be used in combination with the estrogenic compound. The progestional agent is administered in a daily dose, not specified.

WO 04/091535 discloses methods for treating endometrial hyperplasia and vasomotor symptoms comprising administering estrogens and progestins, including starting estrogen therapy with a progestional agent at a high dose, and then lowering the dose once therapy has been shown to be effective. The progestional agent is administered daily in a dose of less than 20 mg.

WO 02/055086 describes a method of hormone replacement therapy comprising administration of an estrogen and/or a gestagen comprising an ingestion-free period, wherein either no estrogen and/or gestagen or a much lower estrogen and/or gestagen content than in the ingestion phases are administered. The document describes that a first ingestion period of estrogen and/or gestagen can be followed by a dosage-reduced ingestion period and then a further dosage-reduced ingestion period.

WO 04/019954 describes a method of estrogen replacement for menopausal women comprising administering ultra-low dose estradiol alternating with standard-dose estradiol. Each standard-dose phase and ultra-low dose phase are consisting of 1-4 days. The method further comprises combined administration a progestin, wherein the progestin administration is a standard sequential or continuous administration or an interrupted or pulsed administration.

Despite of the existing estrogen administration regimens as outlined above, there is a continuous need for optimised Estrogen Replacement Therapy including Estrogen Replacement Therapy which balances the benefits with possible risks.

### SUMMARY OF THE INVENTION

In a first aspect, the present invention concerns a method for the treatment of diseases, conditions or symptoms associated with deficient endogenous levels of estrogen in a woman, said method comprising the steps of
(i) administering to said woman a first therapeutically effective amount of an estrogen during a first treatment period;
(ii) after completion of the first treatment period, administering to said woman a second therapeutically effective amount of an estrogen during a second treatment period, where said second therapeutically effective amount of estrogen is less than said first therapeutically effective amount of estrogen; and optionally
(iii) after completion of the second treatment period, administering to said woman a third therapeutically effective amount of an estrogen during a third treatment period, where said third therapeutically effective amount of estrogen is less than said second therapeutically effective amount of estrogen; or
(iv) after completion of the second treatment period repeating step (i) and optionally step ii).

In a further aspect, the present invention concerns a method for the treatment of diseases, conditions or symptoms associated with deficient endogenous levels of estrogen in a woman and for simultaneously protecting the endometrium from adverse effects of estrogen, said method comprising the steps of
(i) administering to said woman a first therapeutically effective amount of an estrogen during a first treatment period, and administering to said woman a therapeutically effective amount of a progestin during one or more sub-periods of said first treatment period;
(ii) after completion of the first treatment period, administering to said woman a second therapeutically effective amount of an estrogen during a second treatment period, where said second therapeutically effective amount of estrogen is less than said first therapeutically effective amount of estrogen, and administering to said woman a therapeutically effective amount of a progestin during one or more sub-periods of said second treatment period; and optionally
(iii) after completion of the second treatment period, administering to said woman a third therapeutically effective amount of an estrogen during a third treatment period, where said third therapeutically effective amount of estrogen is less than said second therapeutically effective amount of estrogen, administering to said woman a therapeutically effective amount of a progestin during one or more sub-periods of said third treatment period; or
(iv) after completion of the second treatment period repeating step (i) and optionally step ii).

Still further aspects of the present invention relates to pharmaceutical preparations comprising daily dosage units suitable for the step-down estrogen therapy described herein. Thus, more particularly, the present invention also relates to a pharmaceutical preparation comprising a number of separately packed and individually removable daily oral dosage units placed into a packaging unit, wherein

(i-a) each of said daily oral dosage units comprises an estrogen in an amount corresponding to a therapeutically equivalent amount of estradiol hemihydrate in the range of from >0.75 to 1.5 mg, preferably in the range of from >0.75 to 1.25 mg, more preferably in the range of from 0.9 to 1.1 mg, most preferably 1 mg; or

(i-b) each of said daily oral dosage units comprises an estrogen in an amount corresponding to a therapeutically equivalent amount of estradiol hemihydrate in the range of from 0.05 to 0.75 mg, preferably in the range of from 0.25 to 0.75 mg, more preferably in the range of from 0.4 to 0.75 mg, even more preferably in the range of from 0.4 to 0.6 mg, most preferably 0.5 mg; or

(i-c) each of said daily oral dosage units comprises an estrogen in an amount corresponding to a therapeutically equivalent amount of estradiol hemihydrate in the range of from 0.05 to <0.4 mg, preferably in the range of from 0.1 to <0.4 mg, more preferably in the range of from 0.2 to <0.4 mg, even more preferably in the range of from 0.25 to 0.35 mg, most preferably 0.3 mg;

and

(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

### DETAILED DESCRIPTION OF THE INVENTION

The present invention is concerned with a method for treating of diseases, conditions or symptoms associated with deficient endogenous levels of estrogen in women. The basic concept behind the present invention is the realisation that a step-down regimen, which comprises administration of a higher first dose of an estrogen followed by administration of a lower second dose of an estrogen once therapy has been effectively established. Another important aspect of the invention is directed to the simultaneous protection of the endometrium from adverse effects of estrogen. This may be achieved by (partly) co-administration of a progestin, such as drospirenone.

Deficient levels of estrogen can occur for a variety of reasons. For example, deficient levels of estrogen may be caused by e.g. natural menopause, peri-menopause, post-menopause, hypogonadism, castration or primary ovarian failure. Low levels of estrogen, irrespective of the cause, lead to an overall decreased quality of life for women. Symptoms, diseases and conditions range from merely being inconvenient to life threatening. The step-down estrogen therapy descried herein provides effective alleviation of all physiological and psychological signs of estrogen deficiency.

Transient symptoms, such as vasomotor signs and psychological symptoms are certainly embodied with the realm of therapy. Vasomotor signs comprise but are not limited to hot flushes, sweating attacks such as night sweats, and palpitations. Psychological symptoms of estrogen deficiency comprise, but are not limited to, insomnia and other sleep conditions, poor memory, loss of confidence, mood changes, anxiety, loss of libido, difficulties in concentration, difficulty in making decisions, diminished energy and drive, irritability and crying spells.

The treatment of the aforementioned symptoms can be associated with the peri-menopausal phase of a woman's life or after, sometimes long time after, menopause. It is anticipated that the step-down estrogen therapy described herein is applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure.

In another embodiment of the invention, the step-down estrogen therapy is used for the treatment of permanent effects of estrogen deficiency. Permanent effects comprise physical changes such as urogenital atrophy, atrophy of the breasts, cardiovascular disease, changes in hair distribution, thickness of hair, changes in skin condition and osteoporosis.

Urogenital atrophy, and conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume, are symptoms thought to be particularly relevant to step-down estrogen therapy described herein. Furthermore, the step-down estrogen therapy is thought to be relevant to other urogenital changes associated with estrogen deficiency, decreases in mucus production, changes in cell population, decreases in glycogen production, decreases in growth of lactobacilli or increases in growth of streptococci, staphylococci, or coliform bacilli. Other associated changes that are thought to be preventable by the step-down estrogen therapy described herein are those that may render the vagina susceptible to injury or infection, such as exudative discharges, vaginitis, and dyspareunia. Furthermore, infections of the urinary tract and incontinence are other common symptoms associated with lowered estrogen levels.

Other embodiments of the invention include the prevention or alleviation of physical changes associated with estrogen deficiency, such as changes in the skin, changes in hair distribution, thickness of hair, atrophy of the breasts, or osteoporosis.

The prevention and management of osteoporosis, most notably post-menopausal osteoporosis, is a particularly interesting embodiment of the invention. Furthermore, bone demineralisation, reduction of bone mass and density, thinning and interruption of trabeculae, and/or consequent increase in bone fractures or bone deformations are thought to be particularly relevant. The prophylactic treatment of osteoporosis is an interesting therapeutic application of the invention.

A particularly interesting embodiment of the invention is directed to lessening the frequency, persistence, duration and/or severity of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis.

Another interesting embodiment of the invention is directed to treatment of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis.

In the present context, the term a “first therapeutically effective amount”, when used in connection with estrogen treatment, means an amount of the estrogen that is sufficient to lessening the frequency, persistence, duration and/or severity of the symptoms, such as hot flushes, associated with deficient endogenous levels of estrogen. Preferably, the “first therapeutically effective amount” is capable of effectively treat, and hence remove, the symptoms, such as hot flushes, associated with deficient endogenous levels of estrogen.

Likewise, the term a “second therapeutically effective amount”, when used in connection with estrogen treatment, means an amount of the estrogen that is sufficient to at least lessening the frequency, persistence, duration and/or severity of the symptoms, such as hot flushes, associated with deficient endogenous levels of estrogen. Preferably, the “second therapeutically effective amount” is capable of effectively treat, and hence remove, the symptoms, such as hot flushes, associated with deficient endogenous levels of estrogen. More preferably, the “second therapeutically effective amount” is capable of maintaining the woman subject to the estrogen treatment free of symptoms, such as hot flushes, associated with deficient endogenous levels of estrogen and/or the “second therapeutically effective amount” is capable of preventing relapse of symptoms, such as hot flushes, associated with deficient endogenous levels of estrogen.

The term a “third therapeutically effective amount”, when used in connection with estrogen treatment, means an amount of the estrogen that is sufficient to at least lessening the frequency, persistence, duration and/or severity of the symptoms, such as hot flushes, associated with deficient endogenous levels of estrogen. Preferably, the “third therapeutically effective amount” is capable of effectively treat, and hence remove, the symptoms, such as hot flushes, associated with deficient endogenous levels of estrogen. More preferably, the “third therapeutically effective amount” is capable of maintaining the woman subject to the estrogen treatment free of symptoms, such as hot flushes, associated with deficient endogenous levels of estrogen and/or the “third therapeutically effective amount” is capable of preventing relapse of symptoms, such as hot flushes, associated with deficient endogenous levels of estrogen.

The term “first treatment period” as used herein, refers to a period of estrogen therapy where the woman is treated continuously, e.g. daily, with a first therapeutically effective amount of estrogen. The “first treatment period” is continued until the frequency, persistence, duration and/or severity of the symptoms, such as hot flushes, associated with deficient endogenous levels of estrogen has been lessened. Preferably, the “first treatment period” is continued until the symptoms, such as hot flushes, associated with deficient endogenous levels of estrogen, have been effectively treated.

Likewise, the term “second treatment period” as used herein, refers to a period of estrogen therapy where the woman is treated continuously, e.g. daily, with a second therapeutically effective amount of estrogen. The “second treatment period” is continued until the frequency, persistence, duration and/or severity of the symptoms, such as hot flushes, associated with deficient endogenous levels of estrogen has been lessened. Preferably, the “second treatment period” is continued until the symptoms, such as hot flushes, associated with deficient endogenous levels of estrogen, have been effectively treated and will not return.

The term “third treatment period” as used herein, refers to a period of estrogen therapy where the woman is treated continuously, e.g. daily, with a third therapeutically effective amount of estrogen. The “third treatment period” is continued until the symptoms, such as hot flushes, associated with deficient endogenous levels of estrogen, have been effectively treated and will not return.

The term “treatment period”, when is used herein, refers to all of the various treatment periods defined above, i.e. to the “first treatment period”, to the “second treatment period” and to the “third treatment period”. Accordingly, when the term “treatment period” is used herein, all statement and details given in that connection apply equally to the first, second and third treatment period.

The term “estrogen” is meant to encompass all compounds (natural or synthetic, steroidal or non-steroidal compounds) exhibiting estrogenic activity. Such compounds encompass natural and synthetic estradiol and its derivatives; conjugated estrogens; estrogen receptor specific agonists; and non-steroidal compounds exhibiting estrogenic activity. The term is further meant to encompass all isomeric and physical forms of the estrogens including hydrates, such as a hemihydrate; solvates; salts; and complexes, such as complexes with cyclodextrins. A preferred estrogen is estradiol and therapeutically acceptable derivatives thereof.

By the term “conjugated estrogen” is meant the natural conjugated estrogens, such as estrone and equilin and others obtained from pregnant mare urine. Conjugated estrogens are also made synthetically. Examples of synthetically produced estrogens include estropipate and ethinyl estradiol. Further, the term “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include 17-α and β-dihydroequilin, equilenin, 17-α and β-dihydroequilenin, estrone, and their sodium sulfate esters.

When used herein, the term “therapeutically acceptable derivative of estradiol” refers to esters, such as sulfate esters, of estradiol; salts of estradiol and estradiol esters, such as sodium salts, e.g. sodium salts of sulfate esters; as well as other derivatives known in the art. Typically, an ester of estradiol is in the 3-position or 7-position of estradiol. Specific examples of typical esters of estradiol include estradiol valerate, estradiol acetate, estradiol propionate, estradiol enantate, estradiol undecylate, estradiol benzoate, estradiol cypionate, estradiol sulfate, estradiol sulfamate, as well as salts thereof.

The term “estradiol” is intended to mean that the estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate.

In the present context, the term “progestin” covers synthetic progestagens (also sometimes termed progestogens or gestagens). Thus, the term “progestin” covers hormone compounds which exert anti-estrogenic (counteracting the effects of estrogens in the body) and anti-gonadotropic (inhibiting the production of sex steroids and gonads) properties. Progestins are classified according to the structure as C-19 and C-21 progestins, where the C-19 progestins are derived from testosterone and the C-21 progestins are derived from progesterone. Specific examples of progestins include, but is not limited to, progestins selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone. A particular preferred progestin is drospirenone.

The term “therapeutically equivalent amount of ethinyl hemihydrate”, means that other estrogens are administered in amounts which give rise to the same therapeutic effect as does the specified amount of estradiol hemihydrate. Likewise, the term “therapeutically equivalent amount of drospirenone” means that other progestins are administered in amounts which give rise to the same therapeutic effect as does the specified amount of drospirenone. It is routine for those skilled in the art to determine therapeutically equivalent amounts or dosages of such other estrogens and/or progestins when the effective dose of estradiol hemihydrate and/or drospirenone is known. For example, the paper of Timmer and Geurts provides guidance of how equivalent doses may be determined (see “Bioequivalence assessment of three different estradiol formulations in postmenopausal women in an open, randomized, single-dose, 3-way cross-over” in European Journal of Drug Metabolism and Pharmacokinetics, 24(1):47-53, 1999). Moreover, reference is made to EP 1 253 607 which provides a detailed description of therapeutically equivalent amounts of ethinyl estradiol and estradiol on the one hand, and various progestins on the other hand. For further details concerning determination of dose equivalents of various estrogens and progestins, reference is made to “Probleme der Dosisfindung: Sexualhormone” [Problems of Dose-Finding: Sex Hormones]; F. Neumann et aL in “Arzneimittelforschung” (Pharmaceutical Agent Research) 27, 2a, 296-318 (1977), as well as to “Aktuelle Entwicklungen in der hormonalen Kontrazeption” [Current Developments in Hormonal Contraception]; H. Kuhl in Gynäkologe” [Gynecologist] 25: 231-240 (1992).

The terms “pre-menopause”, “peri-menopause”, “menopause” and “post-menopause” are used in their conventional meaning, e.g. as defined on page 9 of “The Controversial Climateric”; P. A. van Keep et al. Ed., MTP Press (1981). More particularly, the term “menopause” is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold (a bleeding threshold), the ovaries can no longer produce mature follicles and sex hormones. The ability to reproduce capability ends with menopause. The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

As indicated above, the present invention relates in a first aspect to the use of an estrogen for the manufacture of a medicament for the treatment of diseases, conditions or symptoms associated with deficient endogenous levels of estrogen in a woman, wherein the administration pattern of said medicament comprises:
(i) administering to said woman a first therapeutically effective amount of an estrogen during a first treatment period;
(ii) after completion of the first treatment period, administering to said woman a second therapeutically effective amount of an estrogen during a second treatment period, where said second therapeutically effective amount of estrogen is less than said first therapeutically effective amount of estrogen; and optionally
(iii) after completion of the second treatment period, administering to said woman a third therapeutically effective amount of an estrogen during a third treatment period, where said third therapeutically effective amount of estrogen is less than said second therapeutically effective amount of estrogen; or
(iv) after completion of the second treatment period repeating step (i) and optionally step ii).

A particular treatment period (e.g. the first treatment period) does not necessarily need to be immediately followed by another treatment period (e.g. the second treatment period), i.e. a treatment-free period may be included between the various treatment periods. However, in a preferred embodiment of the invention the treatment is continued in such a way that the second treatment period follows immediately after the first treatment period, i.e. it is generally preferred that no treatment-free periods are included between the first and the second treatment period, and between the second and the third treatment period.

Step i)—First Treatment Period

In a preferred embodiment of the invention, the estrogen is administered orally during the first treatment period. Preferably, the estrogen is administered orally and once daily during the first treatment period.

The amount of estrogen to be administered during the first treatment period will depend on the actual clinical situation, i.e. the severity of symptoms such as vasomotor symptoms experienced by the woman, the woman's age, the clinical record of the woman, etc. In general, however, the amount of estrogen to be administered once daily during the first treatment period typically corresponds to a therapeutically equivalent amount of estradiol hemihydrate of from >0.75 to 1.5 mg per day, preferably in the range of from >0.75 to 1.25 mg per day, more preferably in the range of from 0.9 to 1.1 mg per day, most preferably about 1 mg per day. While the administered amount of estrogen may be varied within the ranges specified above during the first treatment period, it will be understood that the administered amount of estrogen is preferably the same throughout the first treatment period.

The first treatment period is continued until the frequency, persistence, duration and/or severity of the symptoms associated with deficient endogenous levels of estrogen have been lessened, but is preferably continued until the symptoms associated with deficient endogenous levels of estrogen have been effectively treated. Such symptoms will typically be the vasomotor symptoms, such as hot flushes; sweating attacks, such as night sweats; and palpitations, or a combination thereof. Other symptoms which may be taken into consideration are psychological symptoms, such as insomnia and other sleep conditions; poor memory; loss of confidence; mood changes; anxiety; loss of libido; difficulties in concentration, difficulty in making decisions; diminished energy and drive; irritability; and crying spells. Typically, the physician and/or the patient herself will evaluate the efficiency of the treatment by assessing the reduction or disappearance of vasomotor symptoms, in particular hot flushes.

As will be understood from the above, it may be difficult to give exact guidelines regarding the actual duration of the first treatment period since the duration of the first treatment period will be dependent on the patient's response to the treatment, the amount of estrogen administered, the severity of the symptoms, etc. However, the first treatment period will typically be continued for a period of from 1×28 to 24×28 days. For example, the first treatment period may be from 2×28 to 24×28 days, 3×28 to 24×28 days, 3×28 to 18×28 days, 3×28 to 12×28 days or 3×28 to 9×28 days. Thus, the first treatment period may be continued for 1×28 days, 2×28 days, 3×28 days, 4×28 days, 5×28 days, 6×28 days, 7×28 days, 8×28 days, 9×28 days, 10×28 days, 11×28 days, 12×28 days, 13×28 days, 14×28 days, 15×28 days, 16×28 days, 17×28 days, 18×28 days, 19×28 days, 20×28 days, 21×28 days, 22×28 days, 23×28 days or 24×28 days.

In another embodiment of the invention, the estrogen is administered transdermally during the first treatment period. Transdermal administration of estrogens by means of patches is known in connection with treatment of estrogen deficiencies. Accordingly, the estrogen to be administered transdermally during the first treatment period may be formulated in any transdermal delivery system known in the art, which is capable of providing the desired release of the estrogen. One example of a commercially available estrogen-containing transdermal delivery system is the Menostar® patch marketed by Berlex, USA.

If the estrogen is administered transdermally, the amount of estrogen to be administered during the first treatment period typically corresponds to a therapeutically equivalent amount of estradiol hemihydrate of from >37.5 to 75 μg per day, preferably in the range of from >37.5 to 62.5 μg per day, more preferably in the range of from 45 to 55 μg per day, most preferably about 50 μg per day. The duration of the first treatment period, including the assessments of when to move on to the second treatment period, is the same as described above in connection with oral administration of the estrogen.

Step ii)—Second Treatment Period

In a preferred embodiment of the invention, the estrogen is administered orally during the second treatment period. Preferably, the estrogen is administered orally and once daily during the second treatment period.

In a similar way as described above in connection with the first treatment period, the amount of estrogen to be administered during the second treatment period will depend on the actual clinical situation, i.e. the severity of symptoms experienced by the woman, the woman's age, the clinical record of the woman, etc. In general, however, the amount of estrogen to be administered once daily during the second treatment period typically corresponds to a therapeutically equivalent amount of estradiol hemihydrate of from 0.05 to 0.75 mg per day, preferably in the range of from 0.25 to 0.75 mg per day, more preferably in the range of from 0.4 to 0.75 mg per day, even more preferably in the range of from 0.4 to 0.6 mg per day, most preferably about 0.5 mg per day. While the administered amount of estrogen may be varied within the ranges specified above during the second treatment period, it will be understood that the administered amount of estrogen is preferably the same throughout the second treatment period.

The second treatment period is continued until the frequency, persistence, duration and/or severity of the symptoms associated with deficient endogenous levels of estrogen have, at least, been lessened, but is preferably continued until the symptoms associated with deficient endogenous levels of estrogen have been effectively treated and will not return. Such symptoms will typically be the vasomotor symptoms, such as hot flushes; sweating attacks, such as night sweats; and palpitations, or a combination thereof. Other symptoms which may be taken into consideration are psychological symptoms, such as insomnia and other sleep conditions; poor memory; loss of confidence; mood changes; anxiety; loss of libido; difficulties in concentration, difficulty in making decisions; diminished energy and drive; irritability; and crying spells. Typically, the physician and/or the patient herself will evaluate the efficiency of the treatment by assessing the reduction or disappearance of vasomotor symptoms, in particular hot flushes.

As will be understood from the above, it may be difficult to give exact guidelines regarding the actual duration of the second treatment period since the duration of the second treatment period will be dependent on the patient's response to the treatment, the amount of estrogen administered, the severity of the symptoms, etc. However, the second treatment period will typically be continued for a period of from 1×28 to 36×28 days. For example, the second treatment period may be from 2×28 to 36×28 days, 3×28 to 36×28 days, 3×28 to 24×28 days, 3×28 to 18×28 days, 3×28 to 12×28 days or 3×28 to 9×28 days. Thus, the second treatment period may be continued for 1×28 days, 2×28 days, 3×28 days, 4×28 days, 5×28 days, 6×28 days, 7×28 days, 8×28 days, 9×28 days, 10×28 days, 11×28 days, 12×28 days, 13×28 days, 14×28 days, 15×28 days, 16×28 days, 17×28 days, 18×28 days, 19×28 days, 20×28 days, 21×28 days, 22×28 days, 23×28 days or 24×28 days, 25×28 days, 26×28 days, 27×28 days, 28×28 days or 29×28 days, 30×28 days, 31×28 days, 32×28 days, 33×28 days, 34×28 days, 35×28 days or 36×28 days.

In another embodiment of the invention, the estrogen is administered transdermally during the second treatment period. If the estrogen is administered transdermally, the amount of estrogen to be administered during the second treatment period typically corresponds to a therapeutically equivalent amount of estradiol hemihydrate of from 2.5 to 37.5 μg per day, preferably in the range of from >12.5 to 37.5 μg per day, more preferably in the range of from 20 to 37.5 μg per day, even more preferably in the range of from 20 to 30 μg per day, most preferably about 25 μg per day. The duration of the second treatment period, including the assessments of when to move on to the third treatment period or when to terminate treatment, is the same as described above in connection with oral administration of the estrogen.

Step iii)—Third Treatment Period

In a preferred embodiment of the invention, the estrogen is administered orally during the third treatment period. Preferably, the estrogen is administered orally and once daily during the second treatment period.

In a similar way as described above in connection with the first and second treatment periods, the amount of estrogen to be administered during the third treatment period will depend on the actual clinical situation, i.e. the severity of symptoms experienced by the woman, the woman's age, the clinical record of the woman, etc. In general, however, the amount of estrogen to be administered once daily during the third treatment period typically corresponds to a therapeutically equivalent amount of estradiol hemihydrate of from 0.05 to <0.4 mg per day, preferably in the range of from 0.1 to <0.4 mg per day, more preferably in the range of from 0.2 to <0.4 mg per day, even more preferably in the range of from 0.25 to 0.35 mg per day, most preferably about 0.3 mg per day. While the administered amount of estrogen may be varied within the ranges specified above during the third treatment period, it will be understood that the administered amount of estrogen is preferably the same throughout the third treatment period.

The third treatment period is continued until the frequency, persistence, duration and/or severity of the symptoms associated with deficient endogenous levels of estrogen have, at least, been lessened, but is preferably continued until the symptoms associated with deficient endogenous levels of estrogen have been effectively treated and will not return. Such symptoms will typically be the vasomotor symptoms, such as hot flushes; sweating attacks, such as night sweats; and palpitations, or a combination thereof. Other symptoms which may be taken into consideration are psychological symptoms, such as insomnia and other sleep conditions; poor memory; loss of confidence; mood changes; anxiety; loss of libido; difficulties in concentration, difficulty in making decisions; diminished energy and drive; irritability; and crying spells. Typically, the physician and/or the patient herself will evaluate the efficiency of the treatment by assessing the reduction or disappearance of vasomotor symptoms, in particular hot flushes.

As will be understood from the above, it may be difficult to give exact guidelines regarding the actual duration of the third treatment period since the duration of the third treatment period will be dependent on the patient's response to the treatment, the amount of estrogen administered, the severity of the symptoms, etc. However, the third treatment period will typically be continued for a period of from 1×28 to 48×28 days. For example, the third treatment period may be from 2×28 to 48×28 days, 2×28 to 36×28 days, 2×28 to 24×28 days, 3×28 to 24×28 days, 3×28 to 18×28 days, 3×28 to 12×28 days or 3×28 to 9×28 days. Thus, the third treatment period may be continued for 1×28 days, 2×28 days, 3×28 days, 4×28 days, 5×28 days, 6×28 days, 7×28 days, 8×28 days, 9×28 days, 10×28 days, 11×28 days, 12×28 days, 13×28 days, 14×28 days, 15×28 days, 16×28 days, 17×28 days, 18×28 days, 19×28 days, 20×28 days, 21×28 days, 22×28 days, 23×28 days or 24×28 days, 25×28 days, 26×28 days, 27×28 days, 28×28 days or 29×28 days, 30×28 days, 31×28 days, 32×28 days, 33×28 days, 34×28 days, 35×28 days or 36×28 days, 37×28 days, 38×28 days, 39×28 days, 40×28 days, 41×28 days, 42×28 days, 43×28 days, 44×28 days, 45×28 days, 46×28 days, 47×28 days or 48×28 days.

In another embodiment of the invention, the estrogen is administered transdermally during the third treatment period. If the estrogen is administered transdermally, the amount of estrogen to be administered during the third treatment period typically corresponds to a therapeutically equivalent amount of estradiol hemihydrate of from 2.5 to <20 μg per day, preferably in the range of from 5 to <20 μg per day, more preferably in the range of from 10 to <20 μg per day, even more preferably in the range of from 12.5 to 17.5 μg per day, most preferably about 15 μg per day. The duration of the third treatment period, including the assessments of when to terminate the treatment, is the same as described above in connection with oral administration of the estrogen.

Step iv)

As will be understood by the skilled person, a clinical situation may arise where the amount of estrogen administered during the second treatment period turns out be below the therapeutically effective amount in a specific individual. Thus, even though the amount of estrogen administered in the first treatment period effectively reduced or removed the symptoms associated with the deficient endogenous levels of estrogen in said individual and a shift to the second treatment period was found appropriate, it is contemplated that in some cases it will be necessary to discontinue the treatment in the second treatment period due to recurrence of e.g. hot flushes, and to revert to the treatment conditions specified in step i).

Combination with Progestin

It is well-established that exogenous estrogens stimulate the proliferation of the endometrium. In estrogen monotherapy, the opposing effect of progesterone, which terminates proliferation, is absent. The desquamation phase, during which the top layers of the endometrium are shed, does not occur and proliferation of the endometrium occurs to a greater extent than in the phases up to and including the pre-menopausal phase. The result is hyperplasia, a risk factor for endometrial cancer. Combination therapy, also referred to as opposed therapy, is a treatment where a progestin is added to protect the endometrium from hyperplasia. Accordingly, in a preferred embodiment of the invention, in particular in connection with treatment of diseases, conditions or symptoms associated with deficient endogenous levels of estrogen in a woman who has not undergone hysterectomy (a “non-hysterectomized woman”), co-administration of a progestin for one or more sub-periods of the treatment periods is desirable in order to protect the endometrium from adverse effects of caused by the exogenous estrogen.

Thus, In a preferred aspect, the present invention relates to the use of a combination of an estrogen and a progestin for the manufacture of a medicament for the treatment of diseases, conditions or symptoms associated with deficient endogenous levels of estrogen in a woman and for simultaneously protecting the endometrium from adverse effects of estrogen, wherein the administration pattern of said medicament comprises:
(i) administering to said woman a first therapeutically effective amount of an estrogen during a first treatment period, and administering to said woman a therapeutically effective amount of a progestin during one or more sub-periods of said first treatment period;
(ii) after completion of the first treatment period, administering to said woman a second therapeutically effective amount of an estrogen during a second treatment period, where said second therapeutically effective amount of estrogen is less than said first therapeutically effective amount of estrogen, and administering to said woman a therapeutically effective amount of a progestin during one or more sub-periods of said second treatment period; and optionally
(iii) after completion of the second treatment period, administering to said woman a third therapeutically effective amount of an estrogen during a third treatment period, where said third therapeutically effective amount of estrogen is less than said second therapeutically effective amount of estrogen, administering to said woman a therapeutically effective amount of a progestin during one or more sub-periods of said third treatment period; or
(iv) after completion of the second treatment period repeating step (i) and optionally step ii).

As will be understood, all statements made above in connection with the aspect concerning administration of the estrogen also apply to the aspect concerning co-administration of a progestin. Thus, all statements made above in connection with the duration of the various treatment periods, the amount of estrogen to be administered in the various treatment periods, ways of administering the estrogen, preferred estrogens to be administered, etc. apply mutatis mutandis to the aspect concerning co-administration of a progestin.

While it is contemplated to administer the progestin via the transdermal route, it is currently preferred that the progestin is administered orally. Accordingly, in one embodiment of the invention, the estrogen is administered transdermally in the various treatment periods while the progestin is administered orally. However, in a preferred embodiment of the invention the estrogen as well as the progestin are administered orally during the various treatment periods. In another embodiment of the invention, the estrogen as well as the progestin are administered transdermally. As will be discussed in more detail infra the estrogen and the progestin may be administered individually, i.e. in individual dosage units. However, in a preferred embodiment of the invention, the estrogen and the progestin are present in same dosage unit and hence administered simultaneously.

When administered orally, the progestin is preferably administered once daily during the one or more sub-periods where the progestin is actually administered. As will be understood, the progestin is typically only administered for one or more, relatively short, sub-periods of the various treatment periods. Thus, during a specific treatment period, i.e. during the first, second and/or third treatment period, the progestin is typically only administered in sub-periods having a duration of from ¼×28 to 1×28 days, preferably a duration of from ¼×28 to ¾×28 days, most preferably a duration of ½×28 days. Thus, the progestin may be administered for one or more sub-periods during the entire treatment period. As will be understood, the number of sub-periods (i.e. the number of times progestin treatment is commenced) within each treatment period will be highly dependent on the actual duration of the treatment period. Thus, if the treatment period in question is short, it may only be necessary to include a single sub-period of progestin treatment, whereas if the treatment period in question is relative long, it may be necessary to include two, three, or even more, sub-periods of progestin treatment periods within the treatment period in question.

In general, the interval between initiation of sub-periods of progestin treatment (typically having a duration of from ¼×28 to 1×28 days, preferably a duration of from ¼×28 to ¾×28 days, most preferably a duration of ½×28 days), within each treatment period, should typically be in the order of 2×28 days to 9×28 days. For example, the interval between initiation of sub-periods of progestin treatment (typically having a duration of from ¼×28 to 1×28 days, preferably a duration of from ¼×28 to ¾×28 days, most preferably a duration of ½×28 days), within each treatment period, would typically be 2×28 days, 3×28 days, 4×28 days, 5×28 days, 6×28 days, 7×28 days, 8×28 days or 9×28 days, preferably 3×28 days, 4×28 days, 5×28 days, 6×28 days, 7×28 days. Stated differently, during a given treatment period, a first sub-period of progestin treatment may be initiated 2×28 days, 2.5×28 days, 3×28 days, 3.5×28 days, 4×28 days, 4.5×28 days, 5×28 days, 5.5×28 days, 6×28 days, 6.5×28 days, 7×28 days, 7.5×28 days, 8×28 days, 8.5×28 days or 9×28 days after the treatment period in question is initiated. This first sub-period of progestin treatment may then be followed by a second sub-period of progestin treatment 2×28 days, 3×28 days, 4×28 days, 5×28 days, 6×28 days, 7×28 days, 8×28 days or 9×28 days, preferably 3×28 days, 4×28 days, 5×28 days, 6×28 days, 7×28 days after initiation of the first sub-period of progestin treatment. The above-mentioned intervals between sub-periods of progestin treatment may vary within each treatment period and/or may vary between the treatment periods. As will be understood, longer intervals between sub-periods of progestin treatment may be allowed during the second treatment period as compared to the first treatment period as the administered amount of estrogen is lower in the second treatment period as compared to the first treatment period.

Accordingly, in one embodiment of the invention the interval between initiation of sub-periods of progestin treatment within the first treatment period is 3×28 days.

In another embodiment of the invention the interval between initiation of sub-periods of progestin treatment within the second treatment period is 3×28 days.

In yet another embodiment of the invention the interval between initiation of sub-periods of progestin treatment within the third treatment period is 3×28 days.

In a further embodiment of the invention, the interval between initiation of sub-periods of progestin treatment within the first, the second and/or the third treatment period is 3×28 days.

In one embodiment of the invention the interval between initiation of sub-periods of progestin treatment within the first treatment period is 4×28 days.

In another embodiment of the invention the interval between initiation of sub-periods of progestin treatment within the second treatment period is 4×28 days.

In yet another embodiment of the invention the interval between initiation of sub-periods of progestin treatment within the third treatment period is 4×28 days.

In a further embodiment of the invention, the interval between initiation of sub-periods of progestin treatment within the first, the second and/or the third treatment period is 4×28 days.

In one embodiment of the invention the interval between initiation of sub-periods of progestin treatment within the first treatment period is 5×28 days.

In another embodiment of the invention the interval between initiation of sub-periods of progestin treatment within the second treatment period is 5×28 days.

In yet another embodiment of the invention the interval between initiation of sub-periods of progestin treatment within the third treatment period is 5×28 days.

In a further embodiment of the invention, the interval between initiation of sub-periods of progestin treatment within the first, the second and/or the third treatment period is 5×28 days.

In one embodiment of the invention the interval between initiation of sub-periods of progestin treatment within the first treatment period is 6×28 days.

In another embodiment of the invention the interval between initiation of sub-periods of progestin treatment within the second treatment period is 6×28 days.

In yet another embodiment of the invention the interval between initiation of sub-periods of progestin treatment within the third treatment period is 6×28 days.

In a further embodiment of the invention, the interval between initiation of sub-periods of progestin treatment within the first, the second and/or the third treatment period is 6×28 days.

In one embodiment of the invention the interval between initiation of sub-periods of progestin treatment within the first treatment period is 7×28 days.

In another embodiment of the invention the interval between initiation of sub-periods of progestin treatment within the second treatment period is 7×28 days.

In yet another embodiment of the invention the interval between initiation of sub-periods of progestin treatment within the third treatment period is 7×28 days.

In a further embodiment of the invention, the interval between initiation of sub-periods of progestin treatment within the first, the second and/or the third treatment period is 7×28 days.

Progestins to be used in accordance with this particular aspect of the invention will be known to the skilled person. Specific examples of progestins include, but are not limited to, progestins selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone. In the most preferred embodiment of the invention, the progestin is drospirenone.

As indicated above, the progestin should be administered in a therapeutically effective amount, i.e. in an amount which is capable of protecting the endometrium from adverse effects of the estrogen treatment. Thus, during the sub-periods of progestin treatment (typically having a duration of from ¼×28 to 1×28 days, preferably a duration of from ¼×28 to ¾×28 days, most preferably a duration of ½×28 days), the progestin is administered is typically administered in an amount corresponding to a therapeutically equivalent amount of drospirenone of from 0.5 to 5 mg per day, preferably of from 0.5 to 4 mg per day, more preferably of from 1 to 3 mg per day, even more preferably of from 1.5 to 2.5 mg per day, most preferably 2 mg per day.

Pharmaceutical Compositions

As discussed above, the estrogen may be administered transdermally or via the oral route. When the estrogen, in particular estradiol hemihydrate, is administered via the oral route, the estrogen is preferably contained in an oral dosage unit, such as tablets (both swallowable-only and chewable forms), capsules, granules, granules enclosed in sachets, and pills. Hence, the oral dosage unit containing the estrogen, such as estradiol hemihydrate, may be in the form of a tablet, capsule, gelcap, granule, sachet or a pill. In a preferred embodiment of the invention, the oral dosage unit is in the form of a tablet or a capsule, in particular in the form of a tablet. Tablets may conveniently be coated with a suitable film-forming agent, e.g. hydroxypropylmethylcellulose.

The oral dosage unit containing the estrogen, in particular estradoil hemihydrate, may be formulated in any way conventional in the pharmaceutical art. In particular, the oral dosage unit may be formulated by a method comprising providing the estrogen, such as estradiol hemihydrate, in micronized form in said oral dosage unit, or sprayed from a solution onto particles of an inert carrier in admixture with one or more pharmaceutically acceptable excipients that promote dissolution of the estrogen, such as estradiol hemihydrate. Examples of suitable excipients include fillers, such as lactose, glucose or sucrose, sugar alcohols such as mannitol, starch such as corn or potato starch or modified starch; lubricants such as talc or magnesium stearate; and binders such as polyvinylpyrrolidone, cellulose derivatives, carboxymethyl cellulose, hydroxypropyl cellulose, hydroxypropylmethyl cellulose, methyl cellulose, or gelatin.

With respect to the estrogen, which may be a sparingly soluble substance, it is an advantage to provide it in micronized form or sprayed from a solution, e.g. in ethanol, onto the surface of inert carrier particles, such as described in EP 1 257 280. This has the added advantage of facilitating a more homogenous distribution of the estrogen throughout the composition. When the estrogen, such as estradiol hemihydrate, is provided in micronized form, it preferably has the following particle size distribution as determined under the microscope: 100% of the particles have a diameter of ≦15.0 μm, 99% of the particles have a diameter of ≦12.5 μm, 95% of the particles have a diameter of ≦10.0 μm, and 50% of the particles have a diameter of ≦3.0 μm.

As discussed previously, a progestin, such as drospirenone, may be co-administered with the estrogen in one or more sub-periods during the various treatment periods. The progestin, such as drospirenone, may be formulated in a separate oral dosage unit or the progestin, such as drospirenone, may be formulated in the same oral dosage unit as the estrogen, such as estradiol hemihydrate. Either way, the progestin may be directly incorporated in the oral dosage units described above. However, it is preferred that the progestin, in particular drospirenone, is provided in micronised form or is sprayed from a solution onto particles of an inert carrier in admixture with one or more pharmaceutically acceptable excipients that promote dissolution of the progestin. Accordingly, if provided in micronised form the progestin, such as drospirenone, preferably fulfils the same particle size requirements as given above in connection with micronised estrogen. Independently of the particular formulation of the progestin it is preferred, however, that the progestin is formulated in such a way that at least 70% of the progestin, such as drospirenone, is dissolved within 30 minutes when the oral dosage unit is subjected to dissolution testing in 900 ml of water at 37° C. using the USP XXIII Paddle Method II operated at a stirring rate of 50 rpm. Preferably, at least 80% of the progestin, such as drospirenone is dissolved within 20 minutes when tested as described above. Such compositions are described in EP 1 257 280.

Thus, the present invention also relates to pharmaceutical preparations comprising a number of separately packed and individually removable daily oral dosage units placed into a packaging unit. Such preparations can be adapted in such a way that ready-to-use packages for treatment during the individual treatment periods described herein are provided.

Accordingly, in another aspect the present invention relates to a pharmaceutical preparation comprising a number of separately packed and individually removable daily oral dosage units placed into a packaging unit, wherein

(i) each of said daily oral dosage units comprises an estrogen in an amount corresponding to a therapeutically equivalent amount of estradiol hemihydrate in the range of from >0.75 to 1.5 mg, preferably in the range of from >0.75 to 1.25 mg, more preferably in the range of from 0.9 to 1.1 mg, most preferably 1 mg; and

(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

A further aspect of the present invention relates to a pharmaceutical preparation comprising a number of separately packed and individually removable daily oral dosage units placed into a packaging unit, wherein

(i) each of said daily oral dosage units comprises an estrogen in an amount corresponding to a therapeutically equivalent amount of estradiol hemihydrate in the range of from 0.05 to 0.75 mg, preferably in the range of from 0.25 to 0.75 mg, more preferably in the range of from 0.4 to 0.75 mg, even more preferably in the range of from 0.4 to 0.6 mg, most preferably 0.5 mg; and

(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

A still further aspect of the present invention relates to a pharmaceutical preparation comprising a number of separately packed and individually removable daily oral dosage units placed into a packaging unit, wherein

(i) each of said daily oral dosage units comprises an estrogen in an amount corresponding to a therapeutically equivalent amount of estradiol hemihydrate in the range of from 0.05 to <0.4 mg, preferably in the range of from 0.1 to <0.4 mg, more preferably in the range of from 0.2 to <0.4 mg, even more preferably in the range of from 0.25 to 0.35 mg, most preferably 0.3 mg; and

(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

In a similar way as described above, the estrogen is preferably estradiol or a salt, hydrate or a therapeutically acceptable derivative thereof, in particular estradiol hemihydrate.

Likewise, the progestin may be selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone. As discussed previously, the progestin is preferably drospirenone.

A packaging unit comprising the daily dosage units described above may be prepared in a manner analogous to that of making oral contraceptives or hormone replacement regimens. This may for instance be a conventional blister pack or any other form known for this purpose, for instance a pack comprising the appropriate number of dosage units (in this case at least 28, or for particular applications, a multiple of 28) in a sealed blister pack with a cardboard, paperboard, foil or plastic backing and enclosed in a suitable cover. Each blister container may conveniently be numbered or otherwise marked.

When transdermal formulations are considered, they may be prepared in the form of matrices or membranes or as fluid or viscous formulations in oil or hydrogels. For transdermal patches, an adhesive which is compatible with the skin should be included, such as polyacrylate, a silicone adhesive or polyisobutylene, as well as a foil made of, e.g. polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Suitable transdermal formulations may, for instance, be made in a manner analogous to that described in WO 94/04157 for 3-ketodesogestrel. Alternatively, transdermal formulations may be prepared according to a method disclosed in, e.g., B W Barry, “Dermatological Formulations, Percutaneous Absorption”, Marcel Dekker Inc., New York—Basel, 1983, or Y W Chien, “Transdermal Controlled Systemic Medications”, Marcel Dekker Inc., New York—Basel, 1987.

As will understood by the skilled person, transdermal formulations such as estrogen-containing patches will be worn for a certain period of time, e.g. 3, 4, 5, 6, 7 or up till 14 days (which is typically considerably shorter than the first treatment period), after which the patch needs to be replaced with a new one.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of U.S. Provisional Application Ser. No. 60/812,986 filed Jun. 13, 2006 and U.S. Provisional Application Ser. No. 60/908,794 filed Mar. 29, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for the treatment of diseases, conditions or symptoms associated with deficient endogenous levels of estrogen in a woman, said method comprising the steps of
(i) administering to said woman a first therapeutically effective amount of an estrogen during a first treatment period;
(ii) after completion of the first treatment period, administering to said woman a second therapeutically effective amount of an estrogen during a second treatment period, where said second therapeutically effective amount of estrogen is less than said first therapeutically effective amount of estrogen; and optionally
(iii) after completion of the second treatment period, administering to said woman a third therapeutically effective amount of an estrogen during a third treatment period, where said third therapeutically effective amount of estrogen is less than said second therapeutically effective amount of estrogen; or
(iv) after completion of the second treatment period repeating step (i) and optionally step ii).

2. The method according to claim 1, wherein said first therapeutically effective amount of estrogen is administered orally.

3. The method according to claim 2, wherein said first therapeutically effective amount of estrogen is administered once daily during the first treatment period.

4. The method according to claim 1, wherein said estrogen is estradiol or a salt, hydrate or a therapeutically acceptable derivative thereof.

5. The method according to claim 4, wherein said estrogen is estradiol hemihydrate.

6. The method according to claim 3, wherein said first therapeutically effective amount of estrogen corresponds to a therapeutically equivalent amount of estradiol hemihydrate of from >0.75 to 1.5 mg per day.

7. The method according to claim 1, wherein said second therapeutically effective amount of estrogen is administered orally.

8. The method according to claim 7, wherein said second therapeutically effective amount of estrogen is administered once daily during the second treatment period.

9. The method according to claim 1, wherein said estrogen is estradiol or a salt, hydrate or a therapeutically acceptable derivative thereof.

10. The method according to claim 9, wherein said estrogen is estradiol hemihydrate.

11. The method according to claim 8, wherein said second therapeutically effective amount of estrogen corresponds to a therapeutically equivalent amount of estradiol hemihydrate of from 0.05 to 0.75 mg per day.

12. The method according to claim 1, wherein said third therapeutically effective amount of estrogen is administered orally.

13. The method according to claim 12, wherein said third therapeutically effective amount of estrogen is administered once daily during the third treatment period.

14. The method according to claim 1, wherein said estrogen is estradiol or a salt, hydrate or a therapeutically acceptable derivative thereof.

15. The method according to claim 14, wherein said estrogen is estradiol hemihydrate.

16. The method according to claim 13, wherein said third therapeutically effective amount of estrogen corresponds to a therapeutically equivalent amount of estradiol hemihydrate of from 0.05 to <0.4 mg per day.

17. The method according to claim 1, wherein said first treatment period is continued until the frequency, persistence, duration and/or severity of the symptoms, such as hot flushes, associated with deficient endogenous levels of estrogen have been lessened or until the symptoms, such as hot flushes, associated with deficient endogenous levels of estrogen, have been effectively treated.

18. The method according to claim 1, wherein said first treatment period is from 1×28 to 24×28 days.

19. The method according to claim 1, wherein said second treatment period is continued until the frequency, persistence, duration and/or severity of the symptoms, such as hot flushes, associated with deficient endogenous levels of estrogen has been lessened or until the symptoms, such as hot flushes, associated with deficient endogenous levels of estrogen, have been effectively treated and will not return.

20. The method according to claim 1, wherein said second treatment period is from 1×28 to 36×28 days.

21. The method according to claim 1, wherein said third treatment period is continued until the frequency, persistence, duration and/or severity of the symptoms, such as hot flushes, associated with deficient endogenous levels of estrogen has been lessened or until the symptoms, such as hot flushes, associated with deficient endogenous levels of estrogen, have been effectively treated and will not return.

22. The method according to claim 1, wherein said third treatment period is from 1×28 to 48×28 days.

23. A method for the treatment of diseases, conditions or symptoms associated with deficient endogenous levels of estrogen in a woman and for simultaneously protecting the endometrium from adverse effects of estrogen, said method comprising the steps of
(i) administering to said woman a first therapeutically effective amount of an estrogen during a first treatment period, and administering to said woman a therapeutically effective amount of a progestin during one or more sub-periods of said first treatment period;
(ii) after completion of the first treatment period, administering to said woman a second therapeutically effective amount of an estrogen during a second treatment period, where said second therapeutically effective amount of estrogen is less than said first therapeutically effective amount of estrogen, and administering to said woman a therapeutically effective amount of a progestin during one or more sub-periods of said second treatment period; and optionally
(iii) after completion of the second treatment period, administering to said woman a third therapeutically effective amount of an estrogen during a third treatment period, where said third therapeutically effective amount of estrogen is less than said second therapeutically effective amount of estrogen, administering to said woman a therapeutically effective amount of a progestin during one or more sub-periods of said third treatment period; or
(iv) after completion of the second treatment period repeating step (i) and optionally step ii).

24. The method according to claim 23, wherein said first therapeutically effective amount of estrogen is administered orally.

25. The method according to claim 24, wherein said first therapeutically effective amount of estrogen is administered once daily during the first treatment period.

26. The method according to claim 23, wherein said estrogen is estradiol or a salt, hydrate or a therapeutically acceptable derivative thereof.

27. The method according to claim 26, wherein said estrogen is estradiol hemihydrate.

28. The method according to claim 25, wherein said first therapeutically effective amount of estrogen corresponds to a therapeutically equivalent amount of estradiol hemihydrate of from >0.75 to 1.5 mg per day.

29. The method according to claim 23, wherein said second therapeutically effective amount of estrogen is administered orally.

30. The method according to claim 29, wherein said second therapeutically effective amount of estrogen is administered once daily during the second treatment period.

31. The method according to claim 23, wherein said estrogen is estradiol or a salt, hydrate or a therapeutically acceptable derivative thereof.

32. The method according to claim 31, wherein said estrogen is estradiol hemihydrate.

33. The method according to claim 30, wherein said second therapeutically effective amount of estrogen corresponds to a therapeutically equivalent amount of estradiol hemihydrate of from 0.05 to 0.75 mg per day.

34. The method according to claim 23, wherein said third therapeutically effective amount of estrogen is administered orally.

35. The method according to claim 34, wherein said third therapeutically effective amount of estrogen is administered once daily during the third treatment period.

36. The method according to claim 23, wherein said estrogen is estradiol or a salt, hydrate or a therapeutically acceptable derivative thereof.

37. The method according to claim 36, wherein said estrogen is estradiol hemihydrate.

38. The method according to claim 35, wherein said third therapeutically effective amount of estrogen corresponds to a therapeutically equivalent amount of estradiol hemihydrate of from 0.05 to <0.4 mg per day.

39. The method according to 23, wherein said first treatment period is continued until the frequency, persistence, duration and/or severity of the symptoms, such as hot flushes, associated with deficient endogenous levels of estrogen has been lessened or until the symptoms, such as hot flushes, associated with deficient endogenous levels of estrogen, have been effectively treated.

40. The method according to claim 23, wherein said first treatment period is from 1×28 to 24×28 days.

41. The method according to claim 23, wherein said second treatment period is continued until the frequency, persistence, duration and/or severity of the symptoms, such as hot flushes, associated with deficient endogenous levels of estrogen has been lessened or until the symptoms, such as hot flushes, associated with deficient endogenous levels of estrogen, have been effectively treated and will not return.

42. The method according to claim 23, wherein said second treatment period is from 1×28 to 36×28 days.

43. The method according to claim 23, wherein said third treatment period is continued until the frequency, persistence, duration and/or severity of the symptoms, such as hot flushes, associated with deficient endogenous levels of estrogen have been lessened or until the symptoms, such as hot flushes, associated with deficient endogenous levels of estrogen, have been effectively treated and will not return.

44. The method according to claim 23, wherein said third treatment period is from 1×28 to 48×28 days.

45. The method according to claim 23, wherein said progestin is administered orally.

46. The method according to claim 45, wherein said progestin is administered once daily during said one or more sub-periods.

47. The method according to claim 23, wherein said sub-period is from ¼×28 to 1×28 days.

48. The method according to claim 23, wherein said progestin is selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone.

49. The method according to claim 48, wherein said progestin is drospirenone.

50. The method according to claim 23, wherein said therapeutically effective amount of progestin corresponds to a therapeutically equivalent amount of drospirenone of from 0.5 to 5 mg per day.

51. The method according to claim 23, wherein said woman is a post-menopausal woman.

52. The method according to claim 51, wherein said woman is a post-menopausal and non-hysterectomised woman.

53. The method according to claim 1, wherein said deficient levels of estrogen are caused by natural menopause, peri-menopause, post-menopause, hypogonadism, castration, or primary ovarian failure.

54. The method according to claim 1, wherein said diseases, conditions or symptoms are selected from the group consisting of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition, and osteoporosis, including prevention of osteoporosis.

55. A pharmaceutical preparation comprising a number of separately packed and individually removable daily oral dosage units placed into a packaging unit, wherein
(i) each of said daily oral dosage units comprises an estrogen in an amount corresponding to a therapeutically equivalent amount of estradiol hemihydrate in the range of from >0.75 to 1.5 mg, preferably in the range of from >0.75 to 1.25 mg, more preferably in the range of from 0.9 to 1.1 mg, most preferably 1 mg; and
(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

56. A pharmaceutical preparation comprising a number of separately packed and individually removable daily oral dosage units placed into a packaging unit, wherein
(i) each of said daily oral dosage units comprises an estrogen in an amount corresponding to a therapeutically equivalent amount of estradiol hemihydrate in the range of from 0.05 to 0.75 mg, preferably in the range of from 0.25 to 0.75 mg, more preferably in the range of from 0.4 to 0.75 mg, even more preferably in the range of from 0.4 to 0.6 mg, most preferably 0.5 mg; and
(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

57. A pharmaceutical preparation comprising a number of separately packed and individually removable daily oral dosage units placed into a packaging unit, wherein
(i) each of said daily oral dosage units comprises an estrogen in an amount corresponding to a therapeutically equivalent amount of estradiol hemihydrate in the range of from 0.05 to <0.4 mg, preferably in the range of from 0.1 to <0.4 mg, more preferably in the range of from 0.2 to <0.4 mg, even more preferably in the range of from 0.25 to 0.35 mg, most preferably 0.3 mg; and
(ii) a part of said daily oral dosage units further comprises a progestin in an amount corresponding to a therapeutically equivalent amount of drospirenone in the range of from 0.5 to 5 mg, preferably in the range of from 0.5 to 4 mg, more preferably in the range of from 1 to 3 mg, even more preferably in the range of from 1.5 to 2.5 mg, most preferably 2 mg.

58. The method according to claim 23, wherein said deficient levels of estrogen are caused by natural menopause, peri-menopause, post-menopause, hypogonadism, castration, or primary ovarian failure.

59. The method according to claim 23, wherein said diseases, conditions or symptoms are selected from the group consisting of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition, and osteoporosis, including prevention of osteoporosis.
